(19) United States
(12) Patent Application Publication
(10) Pub. No.: US 2019/0105488 A1
(43) Pub. Date: Apr. 11, 2019

(54) PROGRAMMABLE HYDROGEL IONIC CIRCUITS FOR BIOLOGICALLY MATCHED ELECTRONIC INTERFACES

(71) Applicant: Trustees of Tufts College, Medford, MA (US)

(72) Inventors: Siwei Zhao, Waltham, MA (US); Peter Tseng, Saratoga, CA (US); Jonathan Grasman, Tewksbury, MA (US); Yu Wang, Medford, MA (US); Fiorenzo G. Omenetto, Lexington, MA (US); David L. Kaplan, Concord, MA (US)

(21) Appl. No.: 16/153,759

(22) Filed: Oct. 6, 2018

Related U.S. Application Data
(60) Provisional application No. 62/569,313, filed on Oct. 6, 2017.

(51) Int. Cl. A61N 1/05

(52) U.S. Cl. CPC A61N 1/05

(57) ABSTRACT

The present disclosure relates to programmable hydrogel ionic circuits having properties that are advantageous for use in biological systems. In particular, provided herein are programmable hydrogel ionic circuit that exhibit transparency, stretchability, aqueous-based connective interfaces, high-resolution routing of ionic currents between engineered and biological systems, and reduced tissue damage from electrochemical reactions. As described herein, the programmable hydrogel ionic circuits are produced using a combination of microfluidics and aqueous two-phase systems.

### CROSS-REFERENCE TO RELATED APPLICATIONS

This application claims priority to U.S. Provisional Patent Application No. 62/569,313, filed Oct. 6, 2017, which is incorporated by reference as if set forth in its entirety.

### STATEMENT REGARDING FEDERALLY FUNDED RESEARCH

This invention was made with government support under grant EB002520 awarded by the National Institutes of Health. The government has certain rights in the invention.

### BACKGROUND

In some embodiments, the present invention provides, inter alfa, biologically-matched, programmable hydrogel ionic circuits were developed and delivered localized electrical stimulation in biological environments.

An increasing need for wearable and implantable medical devices has driven the demand for electronics that interface with living systems. Recent advances in materials research have enabled the development of more flexible and biocompatible electronic systems for wearable and implantable biomedical applications. However, existing rigid electron conductor-based electronic systems exhibit fundamental mismatches with biological systems. For example, conductive materials used in these bioelectronic devices usually exploit metals, carbon-based materials and conductive polymers. Most of these materials exhibit good biocompatibility and flexibility, but possess fundamental limitations with regard to stretchability and transparency; requiring specialized material designs (e.g., high aspect ratio nanomaterials, specially formulated conductive polymers) or device architectures (e.g., ultrathin coatings, serpentine circuit design) to achieve desired properties. Such requirements significantly increase design complexity, occupy device real estate, and can fundamentally affect the conductivity of the device. Moreover, most existing conductive materials exhibit a mechanical mismatch with human tissues, making them unsuitable for long-term wear and implantable applications. Most importantly, all of the conductive materials used in the devices carry electron currents (in some cases, hole currents), which have to be converted to ion currents at the electrode/electrolyte interfaces through electrochemical reactions in order to deliver stimulation to biological systems. This process inevitably induces local heat (through Joule heating), pH changes, electrode degradation, and the generation of highly reactive chemical species. These reactions can cause pain and damage to biological tissues, an issue especially relevant for long term or high current electrostimulation, such as in applications in neuromuscular stimulation, transcranial direct current stimulation, electroporation, iontophoresis, wound treatment, pain management, and defibrillation. Thus, new options for materials and devices are needed to facilitate a new generation of bio-compatible electronic systems that can avoid heat, reduce adverse biological effects, and prevent local degradation.

### SUMMARY OF THE DISCLOSURE

The present disclosure relates to programmable hydrogel ionic circuits having properties that are advantageous for use in biological systems. In particular, provided herein are programmable hydrogel ionic circuit that exhibit transparency, stretchability, aqueous-based connective interfaces, high-resolution routing of ionic currents between engineered and biological systems, and reduced tissue damage from electrochemical reactions. As described herein, the programmable hydrogel ionic circuits are produced using a combination of microfluidics and aqueous two-phase systems.

In a first aspect, provided herein is a hydrogel ionic circuit, the circuit comprising or consisting essentially of a molded, crosslinked polyethylene glycol (PEG) hydrogel polymer comprising at least two electrode channels separated by a gap, wherein the channels comprise a salt solution; and at least two ports to connect the salt solution electrode channels to a power source. The PEG hydrogel polymer can comprise at least 15% by weight of a high molecular weight PEG. The PEG hydrogel polymer can comprise at least 20% by weight of a high molecular weight PEG. The PEG can be polyethylene glycol dimethacrylate molecular weight 8,000 (PEGMA 8 k). The PEG hydrogel polymer can additionally comprise at least 15% by weight of a low molecular weight PEG. The PEG hydrogel can comprise at least 20% by weight of polyethylene glycol diacrylate molecular weight 700 (PEGDA 700). The salt solution can be a sodium sulfate (Na₂SO₄) solution or a sodium phosphate (Na₂HPO₄) solution. The salt solution can be a saturated salt solution. The circuit can additionally comprise an electronically responsive component, wherein, when a voltage difference is applied between the salt solution electrode channels, an induced current will activate the electronically responsive component. The electronically responsive component can be a light emitting diode (LED) or organic light emitting diode (OLED). The circuit can additionally comprise one or more cells in the gap between the at least two electrode channels. In some cases, at least two faces of the circuit are covered to prevent water evaporation, wherein the ports extend through the cover. The cover can be an electrical insulating material. The circuit can be optically transparent. The circuit can be stretchable. The circuit can additionally comprise an aqueous-based connective interface at the gap between the salt solution electrode channels.

In another aspect, provided herein is a device comprising one or more hydrogel ionic circuits of this disclosure and a power source. The device can comprise a light-emitting diode (LED) or organic light emitting diode (OLED). The device can comprise an aqueous-based connective interface at the gap between the salt solution electrode channels in the circuit.

In a further aspect, provided herein is a method of stimulating tissue comprising or consisting essentially of the steps of: contacting the aqueous-based connective interface of a circuit of this disclosure to a tissue; and applying a voltage difference across the salt solution electrode channels of the circuit, whereby the induced current stimulates the tissue. The tissue can be a tissue in a subject and the circuit is implanted into the subject. The circuit can be incorporated into a device additionally comprising a power source.

In another aspect, provided herein is a method for fabricating a hydrogel ionic circuit comprising or consisting essentially of the steps of: providing a solution comprising at least 15% by weight of a high molecular weight PEG and between about 0.005% and about 5.0% by weight of a photoinitiator on a mold with a raised or grooved channel pattern; photocrosslinking the high molecular weight PEG by exposure to ultra-violet (UV) light to form a PEG hydrogel with a channel pattern; bonding the PEG hydrogel with a channel pattern to a flat PEG hydrogel by exposure to UV light; and introducing a salt solution into channels of the PEG hydrogel via a port. The solution can comprise at least 20% by weight of a high molecular weight PEG. The high molecular weight PEG can be polyethylene glycol dimethacrylate molecular weight 8,000 (PEGMA 8 k). The solution can additionally comprise at least 15% by weight of a low molecular weight PEG. The solution can comprise at least 20% by weight of polyethylene glycol diacrylate molecular weight 700 (PEGDA 700). The salt solution can be a sodium sulfate (Na₂SO₄) solution or a sodium phosphate (Na₂HPO₄) solution. The salt solution can be a saturated salt solution. The photo-initiator can be selected from the group consisting of 2-hydroxy-1-(4-(hydroxyethoxy)phenyl)-2-methyl-1-propanone and dimethoxy-2-phenyl-acetophenone. One or more light-emitting diodes (LEDs) can be added between the PEG hydrogel with a channel pattern and the flat PEG hydrogel prior to bonding.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1J illustrate a hydrogel ionic circuit embodiment based on ATPS. A, the fabrication and working mechanism of hydrogel ionic circuits. B-E: a 3-LED hydrogel ionic display device and the activation of individual LEDs. The channels in B were stained with powder color dyes. Bar is 1 cm. F: Long-term (two weeks) stability of phase separation between sodium sulfate solution and different PEG hydrogels. Four PEG hydrogel formulas were tested: (1) 5% w/w PEGDMA 8 k and 1% w/w irgacure 2959; (2) 20% w/w PEGDMA 8 k and 1% w/w irgacure 2959; (3) 20% w/w PEGDMA 8 k, 20% w/w PEGDA 700 and 1% w/w irgacure 2959; and (4) 20% w/w PEGDMA 8 k, 40% w/w PEGDA 700 and 1% w/w irgacure 2959. The dotted line showed the resistivity of saturated (sat.) sodium sulfate solution. G, the hydrogel ionic display device was stretched up to 50% strain and remained functional. H: 3-by-5 hydrogel ionic display device. The channels were stained with powder color dyes. Bar is 1 cm. I, the 3-by-5 hydrogel ionic display device was placed on Tufts logos to demonstrate the transparency of the device. J: Arabic numerals and English letters were displayed on the hydrogel ionic display device.

FIGS. 2A-2G demonstrate mechanically responsive and re-programmable hydrogel ionic circuits. A-C, hydrogel ionic circuits with single channel showed channel resistance changes in response to mechanical forces, including bending, stretching and pressing. D-F, single LED touch sensor and the activation of LED by pressing the shunt channel. Bar is 1 cm in D. G, 2-by-3 LED touch array mounted on hand. Only the inlet and outlet areas were covered with small acrylic pieces with access holes to ensure the flexibility of the device. The upper left inset showed a sideview of the device conforming to the surface of the hand with the channels stained with powder color dye. The lower right inset showed one LED was activated by pressing the corresponding shunt channel. Bar is 1 cm.

FIGS. 3A-3C demonstrate localized in vitro cellular electrical stimulation using hydrogel ionic electrode array. A, the schematic of the hydrogel ionic electrode array for in vitro cellular electrical stimulation. B, an image of the actual device. The channels were stained with powder color dyes. Jumper wires were inserted in the top acrylic board to establish electrical connections. Bar is 1 cm. C, left: the intracellular calcium fluorescence changes during experiment. Spot 1 was stimulated, while the other spots were at rest. At 20 and 30 minutes (mins), the fluorescence at stimulated spot (#)was significantly different than that at resting spots (*) (p<0.05). Right: the corresponding fluorescent images at time 0 and 30 mins at each spot. A higher fluorescence increase was seen at the stimulated spots. Bar is 100 μm.

FIGS. 4A-4E demonstrate that hydrogel ionic circuits can induce in vivo muscle electrical stimulation and reduce adverse effects associated with stimulation. A, the schematic of the hydrogel ionic stimulator. B, the hydrogel ionic stimulator was placed on rat TA muscle. The channels were stained with powder color dye. Jumper wires were used to establish electrical connections. Bar is 1 cm. C, the TA muscle was stimulated with 1 kHz and 50 Hz pulsed signals using hydrogel ionic stimulator to induce twitching and tetanus, respectively. The twitch and tetanic forces were compared with controls that used standard gold electrodes. * was significantly different than # (p<0.05). D, high-current injection using stainless steel, carbon and hydrogel ionic electrodes showed that the hydrogel ionic electrodes reduced local heating induced by current injection and prevented tissue damage. “+” is anode and “−” is cathode. Black arrows indicated the lesions. The insets showed the surface temperature profile immediately after current injection. Bars are 5 mm. E, hydrogel ionic electrodes using saturated sodium dibasic phosphate solution as the salt phase reduced pH changes induced by electrochemical reactions. “+” is anode and “−” is cathode. Bars are 5 mm.

FIGS. 5A-5B demonstrate that hydrogel ionic circuits can reduce adverse effects associated with stimulation. A, high-current injection in rat muscle tissue using gold electrodes induced significant muscle damage, including discoloration and disintegration of muscle fibers, especially at anode. The upper right inset is an enlarged view of the area of anode damage showing the boundary between damaged tissue and healthy tissue. Bar is 1 mm. B, muscle damage did not occur with hydrogel ionic electrodes, where muscle striations were smooth and continuous. The broken muscle fibers were due to sectioning defects. Bar is 1 mm.

FIG. 6 presents data from a simulation (using COMSOL Multiphysics® software) of geometry-resistance relationship for single-channel hydrogel ionic circuits subject to mechanical stimuli.

FIG. 7 demonstrates long term phase separation stability between PEG hydrogels and Tyrode's buffer, Dulbecco's Modified Eagle's Medium (DMEM) or sodium dibasic phosphate solution. The dotted lines showed the resistivity of Tyrode's buffer, DMEM or saturated (sat.) Na₂HPO₄ solution.

FIG. 8 illustrates step-by-step fabrication of an embodiment of a 3-LED display device.

FIGS. 9A-9C present data from a simulation (using COMSOL Multiphysics® software) of current distribution in a 3-LED display device. (A) To activate the left LED, the blue channel (common ground) was grounded and the green channel was connected to the AC power supply. To reduce current leakage through the yellow channel, the yellow channel was also grounded. (B) To activate the middle LED, the yellow channel was connected to the AC power and the green channel was ground to reduce current leakage. (C) To activate the right LED, the red channel was connected to the AC power. As can be seen from the COMSOL simulations, high current density was concentrated around the activated LEDs. The current density at the resting LEDs was at least 80% lower than that around the activated LEDs.

FIG. 10 demonstrates optical transmittance of PEG hydrogels and saturated sodium sulfate solution. The thickness of the PEG hydrogels and the sodium sulfate solution is 1 mm.

FIG. 11 presents the compressive strength of PEG hydrogel materials. Three different formulas were tested: 1) 20% w/w PEGDMA 8 k and 1% w/w irgacure 2959; 2) 20% w/w PEGDMA 8 k, 20% w/w PEGDA 700, and 1% w/w irgacure 2959; and 3) 20% w/w PEGDMA 8 k, 40% w/w PEGDA 700, and 1% w/w irgacure 2959.

FIG. 12 presents data from a cyclic mechanical test of hydrogel ionic circuits. Hydrogel ionic circuits with single channel prepared with two different PEG hydrogels were tested with cyclic press of 5 cycles. The maximum pressure is 127.1 kPa for 20% PEGDMA 8 k device and 136.9 kPa for 20% PEGDMA 8 k+20% PEGDA 700 device.

FIG. 13 demonstrates testing of a 2-by-3 LED touch sensor array. LEDs could be individually activated by pressing the corresponding shunt channels with little finger.

FIG. 14 presents data from a simulation (using COMSOL Multiphysics® software) of current distribution in hydrogel ionic electrode array for in vitro cellular electrical stimulation.

FIG. 15 demonstrates localized electrical stimulation of SH-SY5Y at different spots—calcium fluorescence changes during experiment. In the 2nd experiment, the intracellular calcium fluorescence at stimulated spot (#) was significantly different than all resting spots (*) at 20 and 30 minutes (mins) (p<0.05). In the 3rd experiment, the intracellular calcium fluorescence at stimulated spot (#) was significantly different than resting spots 1 and 2 (*) at 20 mins, and different than all resting spots (*) at 30 mins (p<0.05). In the 4th experiment, the intracellular calcium fluorescence at stimulated spot (#) was significantly different than all resting spots (*) at all three time points (p<0.05).

FIG. 16 presents data from a simulation (using COMSOL Multiphysics® software) of current distribution in in vivo muscle electrical stimulation. The electrical conductivity of rat muscle used in the simulation was 0.3 S/m (37). As can be seen from the simulation, the current density was concentrated in the small region between the two salt solution channels/electrodes, which allowed localized muscle stimulation.

FIG. 17 demonstrates setup of the in vivo muscle electrical stimulation experiment.

FIG. 18 illustrates a hydrogel ionic electrode setup for current injection in chicken breast and PBS-soaked pH paper.

### DETAILED DESCRIPTION

All of the patents and publications referred to herein are incorporated by reference in their entirety.

The methods and compositions disclosed herein are based at least in part on the inventors' development of salt/polyethylene glycol aqueous two-phase systems to fabricate programmable hydrogel ionic circuits. High-conductivity salt-solution-patterns were stably encapsulated within polyethylene glycol hydrogels using salt/polyethylene glycol phase separation to enable designer electronics tailored to display traits matched to biological systems. These include transparency, stretchability, complete aqueous-based connective interface, high-resolution routing of ionic current between engineered and biological systems, and reduced tissue damage from electrochemical reactions. The potential of such systems was demonstrated by generating a series of functional devices, including multi-pixel light-emitting diode displays, mechanically-adaptable circuits, skin-mounted electronics, and stimulators that delivered localized current to in vitro neuron cultures and in vivo muscles in live animals. Such electronic platforms may form the basis of interlaced, bioelectronic systems into the future.

Herein, aspects of the present invention provide aqueous-stable, hydrogel ionic circuits that were fabricated using a combination of microfluidics and aqueous two-phase systems (ATPS, the phase separation between polyethylene glycol and incompatible salts in aqueous environment). These hydrogel ionic circuits are transparent, stretchable and the circuit design of ionically conductive patterns can be mechanically re-programmed after the circuits are fabricated. Furthermore, certain aspects of the present invention demonstrate the utility of these hydrogel ionic circuits in delivering localized electrical stimulation in biological environments with reduced adverse effects when compared to conventional metal- and carbon-based electrodes.

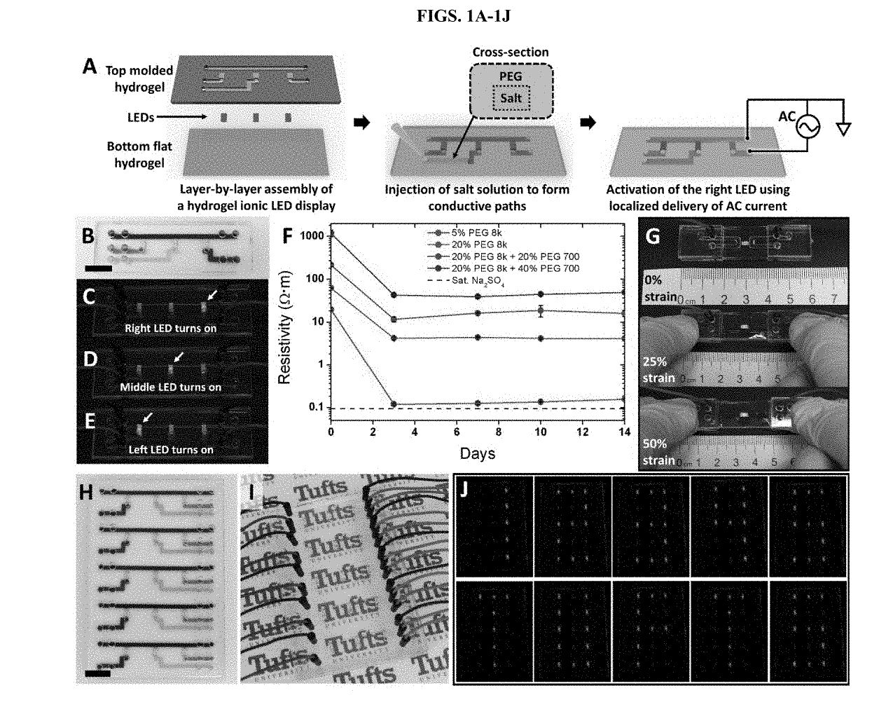

In some embodiments, new hydrogel ionic circuits were developed based on ATPS, a phenomenon discovered more than one century ago, and used primarily for biomolecule separation and purification (20-22). Polyethylene glycol (PEG) is commonly used in ATPS, which can phase separate with various salts, such as sodium sulfate and sodium phosphate (23, 24). The PEG and salt are mixed together in an aqueous solution and centrifuged to allow the two phases to separate. Once the two-phase system forms, the PEG-rich phase has a low salt content and thus low ionic conductivity, while the salt-rich phase is highly conductive. To generate hydrogel ionic circuits based on salt/PEG ATPS, microchannels having desired conductive patterns were molded into photocrosslinked PEG hydrogels using polydimethylsiloxane (PDMS) molds (FIG. 1A). The molded PEG gels were subsequently bonded to a flat PEG gel by UV exposure to close the channels. The channels were perfused with concentrated salt solution to establish paths with high conductivity, which were stably contained in the channels. If a voltage difference is applied between two salt channels separated by a gap, for example the PEG hydrogel itself or cell culture media, the induced current will tend to follow the pattern of the channels and cross the gap at the narrowest part, as electrical current follows the path of least resistance. Electrically-responsive components sitting in the current path, such as an light-emitting diode (LED) encapsulated in the PEG hydrogel, or cells cultured in the medium at the narrowest part of the gap, will be activated by the current (FIGS. 1B-1E).

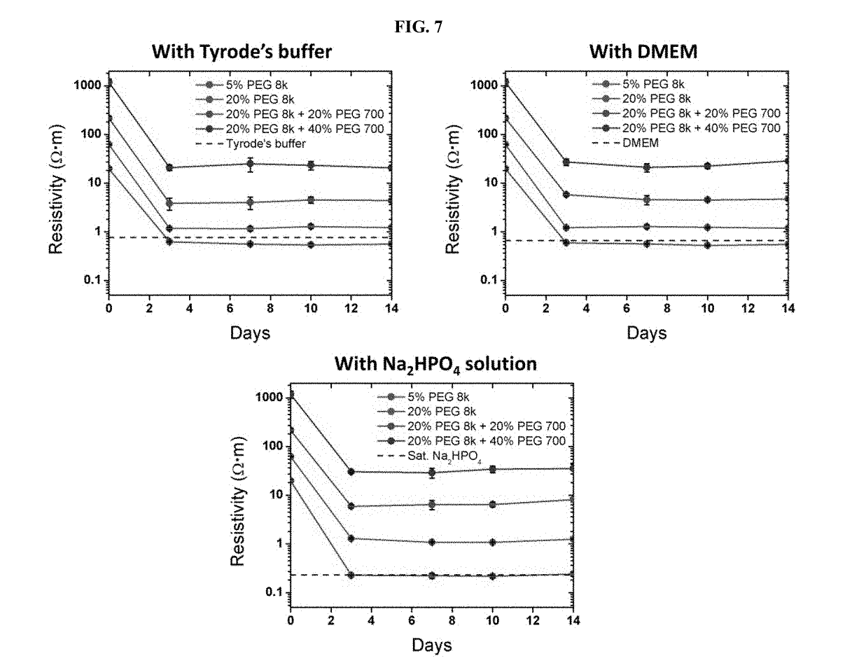

In some embodiments, two phase separation can be important in certain provided hydrogel ionic circuits is the two-phase separation, which is dependent on the species of salt, the molecular weight of PEG and the concentrations of PEG and salt. We have tested the two-phase formation and long-term stability using different PEG polymers and salt-containing media by soaking the PEG hydrogels in media and monitoring their resistivity. A higher PEG concentration led to more stable two-phase separation with higher resistivity contrast between PEG hydrogels and salt media (FIG. 1F, FIG. 7). The addition of low molecular weight PEG also facilitated the formation of the two-phase system.

It will be understood by one of skill in the art that any PEG capable of two-phase separation can be used for the hydrogel ionic circuits and methods provided herein. For instance, PEGs having a molecular weight between about 600 to about 8,000 (e.g., having a MW of about 600, 650, 700, 800, 900, 1000, 1200, 1400, 1600, 1800, 2000, 3000, 4000, 5000, 6000, 7000, 8000 g/mol, inclusive) have been used to form aqueous two-phase systems. By way of non-limiting example, the Examples demonstrate embodiments using PEGs having molecular weights of 700 and 8,000.

It will be understood by one of skill in the art, that changes in resistivity for various tunable circuits may be desired. Therefore, the concentration of the salt solution used in the channels of the hydrogel circuit may be variable. Salts for use in the salt solution include, but are not limited to, sodium chloride (NaCl), sodium sulfate (Na₂SO₄), sodium phosphate (Na₂HPO₄), magnesium chloride (MgCl₂), potassium bromide (KBr), and the like. Among different media, sodium sulfate and dibasic phosphate salt solutions were more efficient for two-phase formation using some of the PEG hydrogels described herein. With cell culture medium or Tyrode's solution (a buffer used for cellular electrical stimulation), higher concentration and lower molecular weight of PEG were required for stable phase separation. Because saturated sodium sulfate solution has the highest resistivity contrast with the PEG hydrogels tested, we chose to use this as the salt phase for Examples described below, unless otherwise noted.

In some embodiments, the PEG hydrogels include at least about 15%, at least about 20%, at least about 25%, or at least about 30% by weight of a high molecular weight PEG. As used herein, “high molecular weight PEG” refers to a polyethylene glycol with a molecular weight greater than 5 kDa (i.e., 5,000 g/mol). For incorporation into photo-crosslinked hydrogels, the high molecular weight PEG is functionalized with one more terminal acrylate groups which are polymerizable by photo-crosslinking. The PEG acrylate may be a PEG diacrylate (PEGDA) or a PEG dimethacrylate (PEGDMA). In some embodiments, the high molecular weight PEG for use in the PEG hydrogel is PEG dimethacrylate, molecular weight 8,000 (PEGDMA 8 k).

In some embodiments, the PEG hydrogels additionally include at least about 15%, at least about 20%, at least about 25%, at least about 30%, at least about 35%, at least about 40%, or at least about 45% by weight of a low molecular weight PEG. As used herein, “low molecular weight PEG” refers to a polyethylene glycol with a molecular weight less than 1,000 g/mol. For incorporation into photo-crosslinked hydrogels, the low molecular weight PEG is functionalized with one more terminal acrylate groups which are polymerizable by photo-crosslinking. The PEG acrylate may be a PEG diacrylate (PEGDA) or a PEG dimethacrylate (PEGDMA). In some embodiments, the low molecular weight PEG for use in the PEG hydrogel is PEG diacrylate, molecular weight 700 (PEGDA 700).

In some embodiments, the hydrogel comprises a biodegradable polymer. As used herein, the term “biodegradable” is used to refer to materials (e.g., polymers) that will degrade over time by the action of enzymes, by hydrolytic action, and/or by other similar mechanisms in the human body. In some cases, biodegradable hydrogels, when introduced into cells or tissues, are broken down by cellular machinery (e.g., enzymatic degradation) or by hydrolysis into components that cells or tissues can either reuse or dispose of without significant toxic effect(s). In certain embodiments, components generated by breakdown of a biodegradable material do not induce inflammation and/or other adverse effects in vivo. In some embodiments, biodegradable materials are enzymatically broken down. For example, a hydrogel can be biodegradable through the use of enzyme labile crosslinkers. Alternatively or additionally, in some embodiments, biodegradable materials are broken down by hydrolysis. In some embodiments, biodegradable polymeric materials break down into their component and/or into fragments thereof (e.g., into monomeric or submonomeric species). In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as silk, albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

In some embodiments, the elastomeric property of PEG hydrogel materials allows the dimensions of the salt solution channels to be altered by external forces (FIG. 11). This allows the hydrogel ionic circuits to be re-programmed after the devices are made, which has potential applications such as blood pressure sensing for health monitoring and touch input panels for human-computer interfaces.

Any method known to one skilled in the art for cross-linking can be used for preparing the hydrogels. In some cases, polymerized using photo-cross-linking methods. Photoinitiators produce reactive free radical species that initiate the cross-linking and/or polymerization of monomers upon exposure to light. Any photoinitiator can be used in the cross-linking and/or polymerization reaction.

In some embodiments, the photoinitiator can be a peroxide (for example, ROOR′), a ketone (for example, RCOR′), an azo compound (i.e. compounds with a —N═N— group), an acylphosphineoxide, a sulfur-containing compound, a quinone. Exemplary photoinitiators include, but are not limited to, acetophenone; anisoin; anthraquinone; anthraquinone-2-sulfonic acid, sodium salt monohydrate; (benzene) tricarbonylchromium; 4-(boc-aminomethyl)phenyl isothiocyanate; benzin; benzoin; benzoin ethyl ether; benzoin isobutyl ether; benzoin methyl ether; benzoic acid; benzophenyl-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diefhylamino)benzophenone; 4,4′-bis(dimethylamino)benzophenone; Michler' s ketone; camphorquinone; 2-chlorothioxanthen-9-one; 5-dibenzosuberenone; (cumene)cyclopentadienyliron(II) hexafluorophosphate; dibenzosuberenone; 2,2-diefhoxyacetophenone; 4,4′-dihydroxybenzophenone; 2,2-dimethoxy2-phenylacetophenone; 4-(dimethylamino)benzophenone; 4,4′-dimethylbenzyl; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methyl benzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizes (Biddle Sawyer); Irgacure 907 (Ciba Geigy); Irgacure 2959 (CIBA Geigy); Irgacure 651 (Ciba Geigy); Darocur 2959 (Ciba Geigy); ethyl-4-N,N-dimethylaminobenzoate (4EDMAB); 1-[-(4-benzoylphenylsulfanyl)phenyl]-2-methyl-2-(4-methylphenylsulfonyl)propan1-one; 1-hydroxy-cyclohexyl-phenyl-ketone; 2,4,6trimethylbenzoyldiphenylphosphine oxide; diphenyl(2,4,6trimethylbenzoyl)phosphine; 2-ethylhexyl-4 dimethylaminobenzoate; 2-hydroxy-2-methyl-1-phenyl-1-propanone; 65% (oligo[2-hydroxy-2-methyl-1-[4-(1methylvinyl)phenyl]propanone] and 35% propoxylated glyceryl triacrylate; benzil dimethyl ketal; benzophenone; blend of benzophenone and a-hydroxy-cyclohexyl-phenylketone; blend of Esacure KIP150 and Esacure TZT; blend of Esacure KIP150 and Esacure TZT; blend of Esacure KIP150 and TPGDA; blend of phosphine oxide, Esacure KIP150 and Esacure TZT; difunctional a-hydroxy ketone; ethyl 4-(dimethylamino)benzoate; isopropyl thioxanthone; 2-hydroxy-2methyl-phenylpropanone; 2,4,6,-trimethylbenzoyldipheny-1-phosphine oxide; 2,4,6-trimethyl benzophenone; liquid blend of 4-methylbenzophenone and benzophenone; oligo(2-hydroxy-2-methyl-1-(4(1-methylvinyl)phenyl)propanone; oligo(2-hydroxy-2-methyl-1-4(1-methylvinyl)phenyl propanone and 2-hydroxy-2-methyl-1-phenyl-1-propanone (monomeric); oligo(2-hydroxy-2-methyl-1-4(1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone. In certain embodiments, the photoinitiator is acetophenone; diphenyl(2,4,6-trimethylbenzoyl)phosphine oxide; 4,4′-dimethoxybenzoin; anthraquinone; anthraquinone-2-sulfonic acid; benzene-chromium(O) tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1 hydroxycyclohexyl phenyl ketone, 50/50 blend; benzophenone-3,3′,4,4′-tetracarboxylic dianhydride; 4-benzoylbiphenyl; 2-benzyl-2-(dimethyl amino)-4′ morpholinobutyrophenone; 4,4′-bis(diethylamino) benzophenone; Michler' s ketone; (±)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4dimethylbenzophenone; diphenyl (2,4,6-trimethylbenzoyl) phosphine oxide/2-hydroxy methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts; 3-mercapto-1-propanol; 11-mercapto-1-undecanol; 1-mercapto-2-propanol; and 3-mercapto-2-butanol, all of which are commercially available from Sigma-Aldrich. In certain embodiments, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1 (4-(1-methylvinyl)phenyl)propanone and 4-methylbenzophenone. In some embodiments, the photoinitiator is dimethoxy-2-phenyl-acetophenone (DMPA), a titanocene, 2-hydroxy-1-(4(hydroxyethoxy)phenyl)-2-methyl-l-propanone, Igracure.

In some embodiments, the initiator is 2-hydroxy-1-(4-(hydroxyethoxy)phenyl)-2-methyl-1-propanone (Irgacure 2959, CIBA Chemicals).

In general, photoinitiators are utilized at concentrations ranging between approximately 0.005% w/v and 5.0% w/v. For example, photoinitiators can be utilized at concentrations of about 0.005% w/v, about 0.01% w/v, about 0.025% w/v, about 0.05% w/v, about 0.075% w/v, about 0.1% w/w, about 0.125% w/v, about 0.25% w/v, about 0.5% w/v, about 0.75% w/v, about 1% w/v, about 1.125% w/v, about 1.25% w/v, about 1.5% w/v, about 1.75% w/v, about 2% w/v, about 2.125% w/v, about 2.25% w/v, about 2.5% w/v, about 2.75% w/v, about 3% w/v, about 3.125% w/v, about 3.25% w/v, about 3.5% w/v, about 3.75% w/v, about 4% w/v, about 4.125% w/v, about 4.25% w/v, about 4.5% w/v, about 4.75% w/v, about 5% w/v or higher, although high concentrations of photo-initiators can be toxic to cells.

Methods other that photo-cross-linking can also be used for preparing hydrogel ionic circuits. For example, cross-linking can be achieved utilizing chemical cross-linking agents, physical cross-linking methods (for example, repeated cycles of freezing and thawing can induce cross-linking of particular polymers), irradiative cross-linking methods, thermal cross-linking methods, ionic cross-linking methods, and the like.

In some embodiments, the circuit comprises an electronically responsive component. The electronically responsive component can be any device, sensor, machine, light, tool, or another circuit that can be activated by an electric current. In some embodiments, the electronically responsive component is a light-emitting diode (LED) or an organic light emitting diode (OLED). Other suitable electronically responsive components include, without limitation, resistors, heaters, energy storage units, low-power display units, and low-power speakers. In some embodiments, the electronically responsive component are cells cultured in the narrowest part of the gap between the salt solution electrode channels.

In some embodiments, the top and bottom of the hydrogel circuit are covered to prevent water evaporation. Covers may be made of any electrical insulating material which itself will not conduct an electric current. Suitable materials include, but are not limited to, acrylic, glass, plastic, biodegradable polymers, ceramic, polypropylene, TeflonTM, nylon, polycarbonate, and polyvinyl chloride. The cover will also include one or more holes or ports to facilitate easy fluid injection and electrical connection with the salt solutions in the channels.

The hydrogel ionic circuits described herein can be assembled into a device. The device can include one or more circuits and a power source. The circuits are connected to the power source via the ports to the salt solution electrode channels. Devices including the circuits described herein may also include one or more electronically responsive comments, such as an LED. The devices may also include an aqueous-based connective interface to connect the circuit to a tissue in vivo or to an in vitro cell culture.

Practical Applications

Circuits and devices described herein may be used to stimulate tissues. The devices and circuits may be fabricated in to skin-mounted electronic stimulators or implantable electronic stimulators. Tissues suitable for stimulation using the devices and circuits described herein include, but are not limited to, muscle tissue (e.g., cardiac muscle, smooth muscle, skeletal muscle), nervous tissues (e.g., brain tissue, spinal cord, nerves), epithelial tissue (e.g., lining of the gastrointestinal tract organs, lining of other hallow organs, surface of the skin), and connective tissue (e.g., fat, tendons).

In some embodiments, circuits and devices described herein may be used to stimulate cardiac tissue and may be fabricated into pacemakers or defibrillators. The pacemakers or defibrillators including the circuits described herein may be implanted into the chest cavity of a subject along with a suitable power source.

In some embodiments, circuits and devices described herein may be used to stimulate skin or skeletal muscle tissue and be fabricated for implantation on or directly below the surface of the skin.

The circuits and devices described herein can also be fabricated into stretchable multi-pixel light-emitting diode (LED)-based display devices that can display an array of Arabic numerals and letters.

The devices and circuits described herein are flexible circuits that are mechanically reconfigurable and it is envisioned that they can be fabricated into skin-mounted electronics that can detect touch.

The term “implantable” as used herein refers to a biocompatible device (e.g., hydrogel ionic circuit-based device) retaining potential for successful placement within a mammal. The expression “implantable device” and expressions of the like as used herein refers to an object implantable through surgery, injection, or other suitable means whose primary function is achieved either through its physical presence or mechanical properties.

Definitions

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The present invention will be more fully understood upon consideration of the following non-limiting Examples. All texts, papers, and patents disclosed herein are hereby incorporated by reference as if set forth in their entirety.

### EXAMPLES

Reference is now made to the following examples, which together with the above descriptions illustrate the invention in a non-limiting fashion.

### Example 1

### Developing Programmable Hydrogel Ionic Circuits for Biologically-Matched Electronic Interfaces

To generate hydrogel ionic circuits based on salt/PEG ATPS, microchannels with desired conductive patterns were molded into photocrosslinked PEG hydrogels using polydimethylsiloxane (PDMS) molds (FIG. 1A). The molded PEG gels were subsequently bonded to a flat PEG gel by UV exposure to close the channels. The channels were perfused with concentrated salt solution to establish paths with high conductivity, which were stably contained in the channels. If a voltage difference is applied between two salt channels separated by a gap, for example the PEG hydrogel itself or cell culture media, the induced current will tend to follow the pattern of the channels and cross the gap at the narrowest part, as electrical current follows the path of least resistance. Electrically-responsive components sitting in the current path, such as an light-emitting diode (LED) encapsulated in the PEG hydrogel, or cells cultured in the medium at the narrowest part of the gap, will be activated by the current (FIGS. 1B-1E).

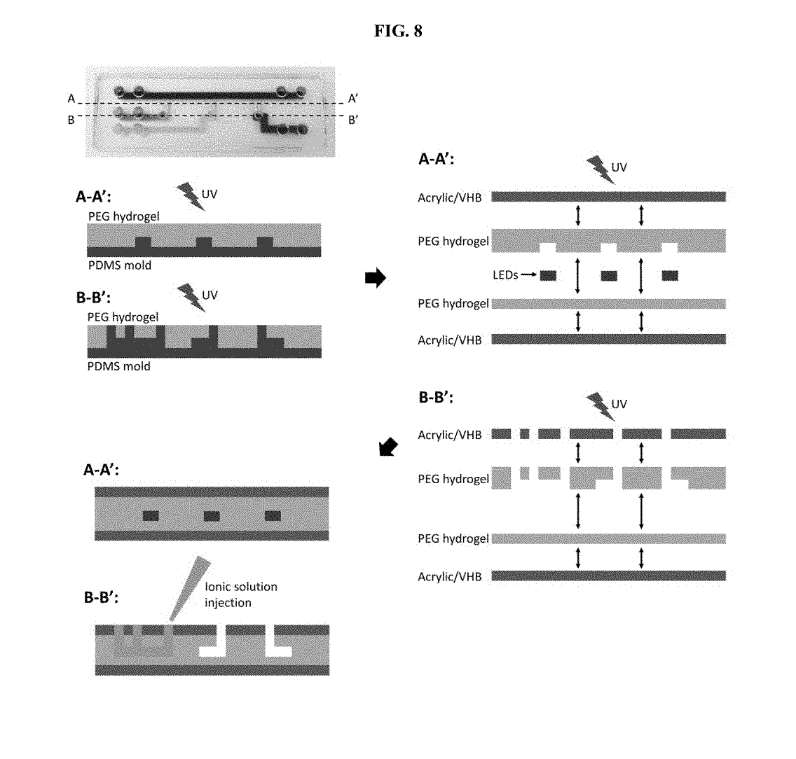

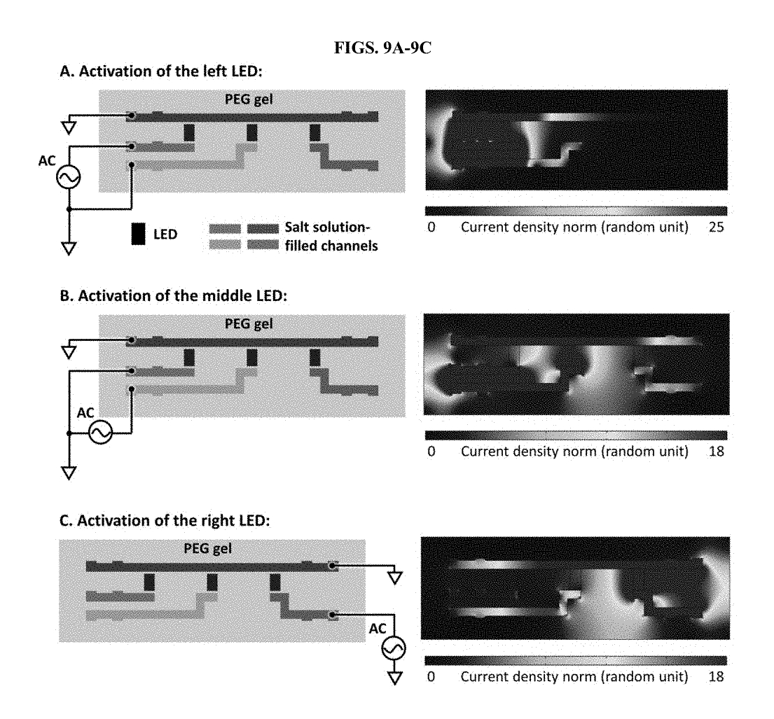

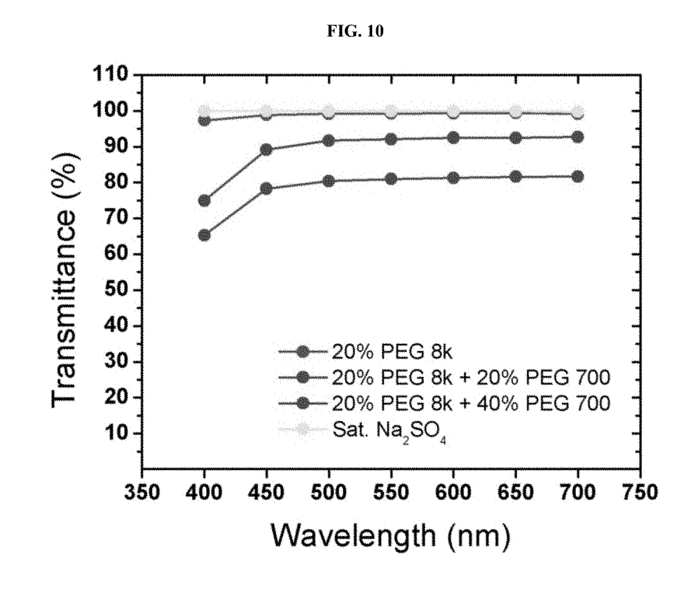

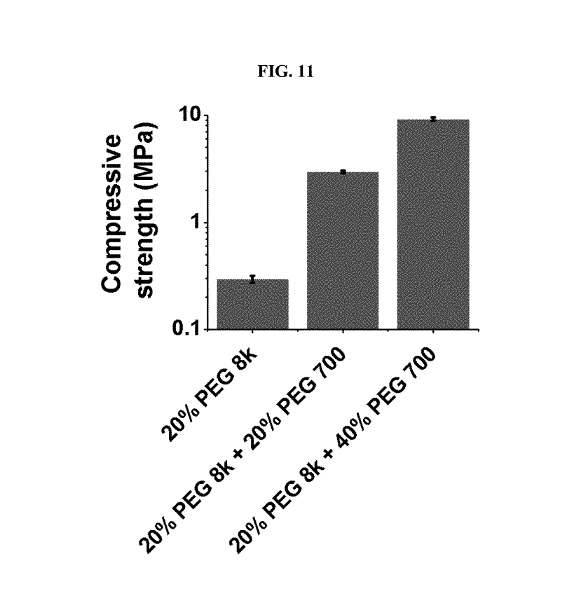

A simple hydrogel ionic display device comprising three LEDs/pixels was fabricated to demonstrate the concept of these hydrogel ionic circuits (FIGS. 1B-1E). This device was prepared with PEG precursor solution consisting of 20% w/w PEG dimethacrylate (PEGDMA, molecular weight=8,000), 20% w/w PEG diacrylate (PEGDA, molecular weight=700) and 1% w/w Irgacure 2959 (FIG. 8 and Materials/Methods). The hydrogel device was sandwiched by two pieces of acrylic board to prevent water evaporation. Access holes were laser cut on the top acrylic board for easy fluid injection and electrical connections. The blue channel served as a common ground; the ground channel. The other three channels (green, yellow, red) were connected to an alternating current (AC) generator for the activation of the LEDs; the power channels. All three LEDs could be individually addressed and activated by injecting AC current into the different power channels (FIGS. 1C-1E and FIG. 9). This result indicates that these hydrogel ionic circuits were able to guide current in an aqueous environment. To demonstrate the design and complexity of these hydrogel ionic circuits, the hydrogel ionic display device was expanded to a 3-by-5, 15-pixel display, which is commonly seen in calculators and clocks and is able to display English letters and Arabic numerals (FIG. 1H). The 3-by-5 display had a similar circuit design as the 3-pixel display with three power channels and one ground channel in each row. Since both the PEG hydrogel and salt solution are transparent, the 3-by-5 display device had high transmittance over the entire visible spectrum (FIG. 1I, FIG. 10). Again, all 15 LEDs could be individually addressed and activated. We successfully used the device to display the logo for Tufts University “TUFTS” and Arabic numbers of “1” to “5” (FIG. 1J). In addition, the hydrogel display devices remained functional under moderate stretch, up to 50%, before the hydrogel failed (FIG. 1G).

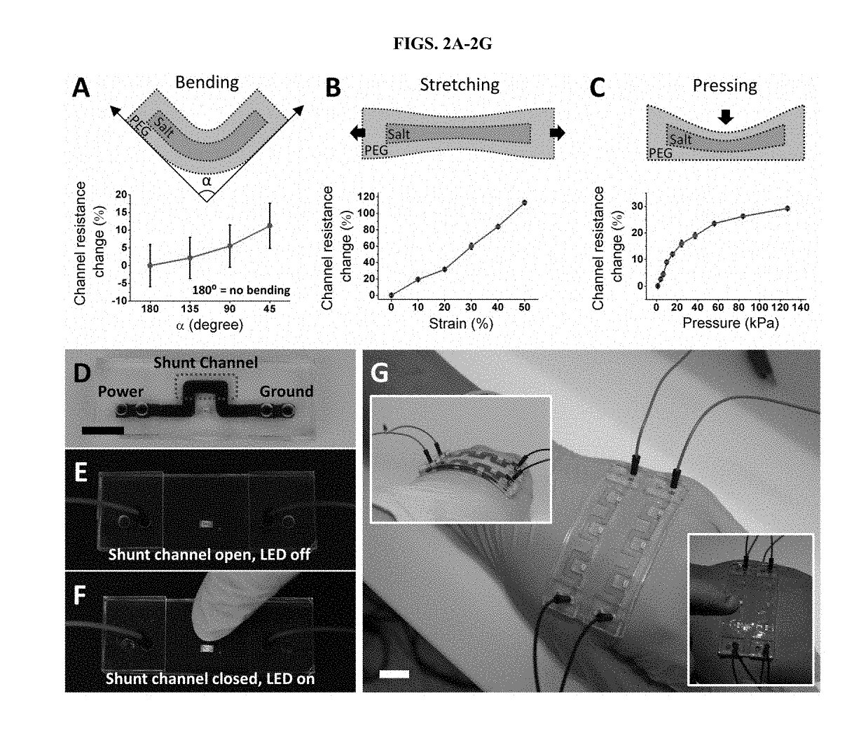

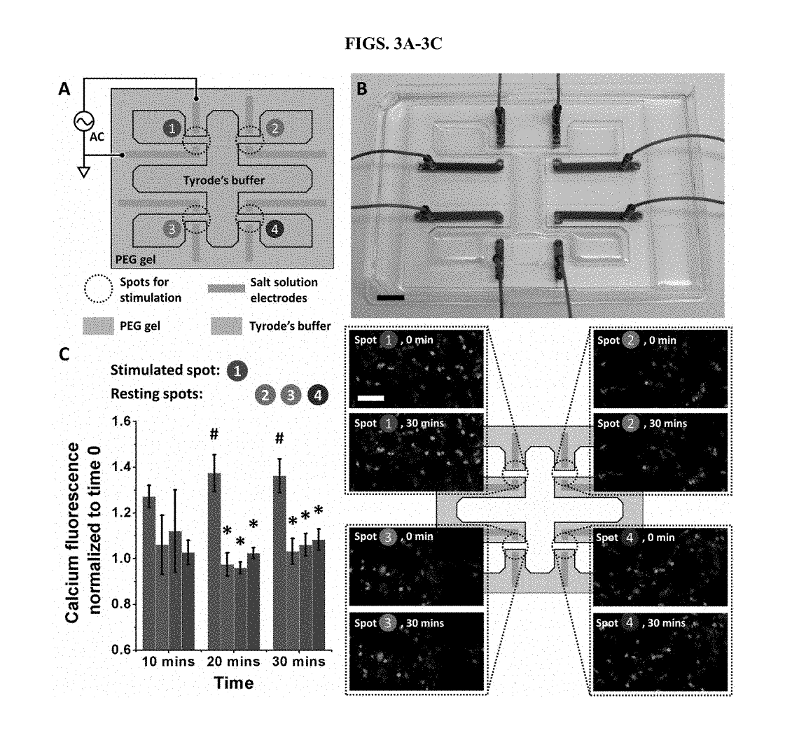

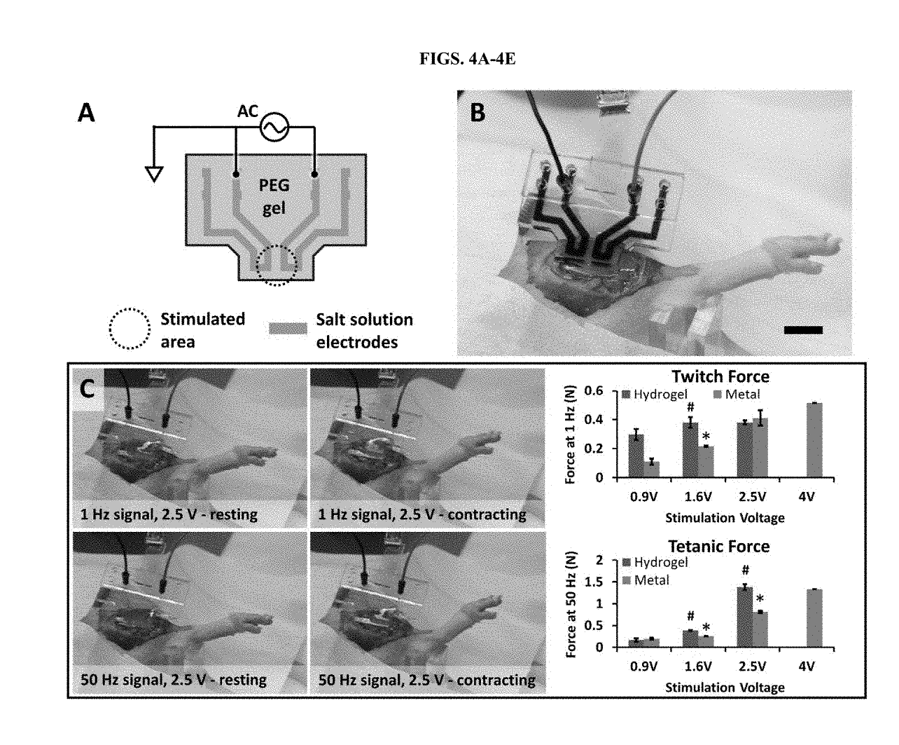

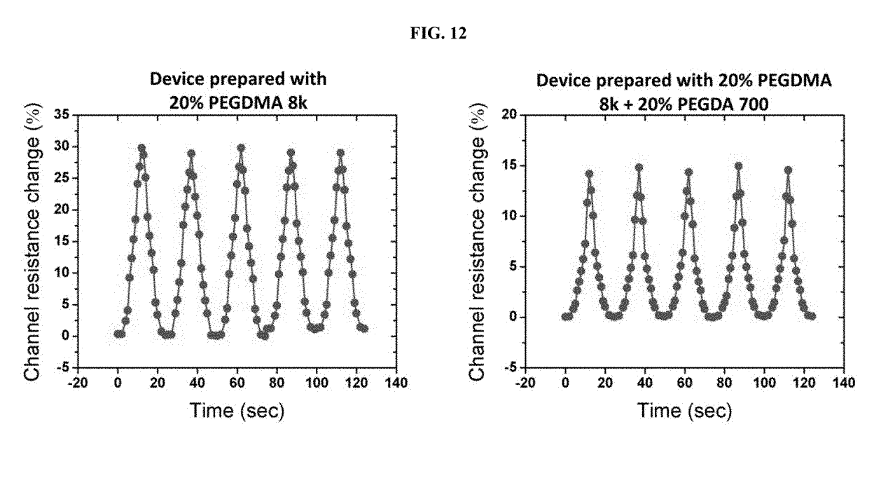

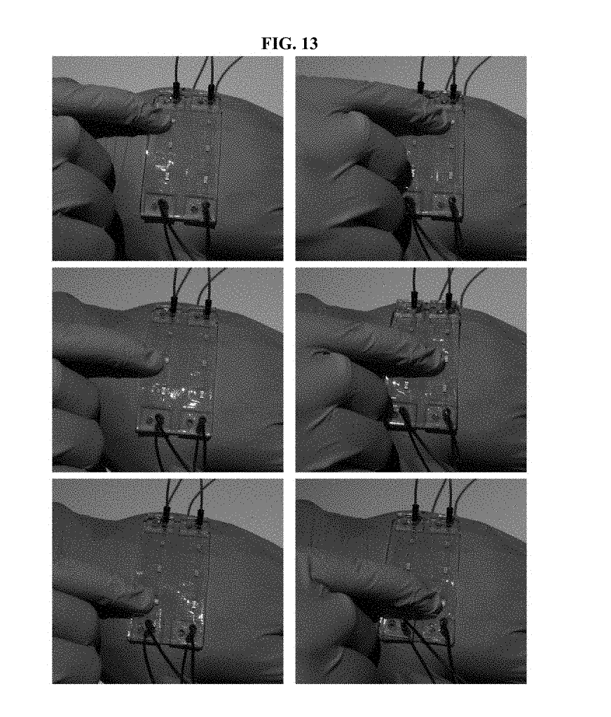

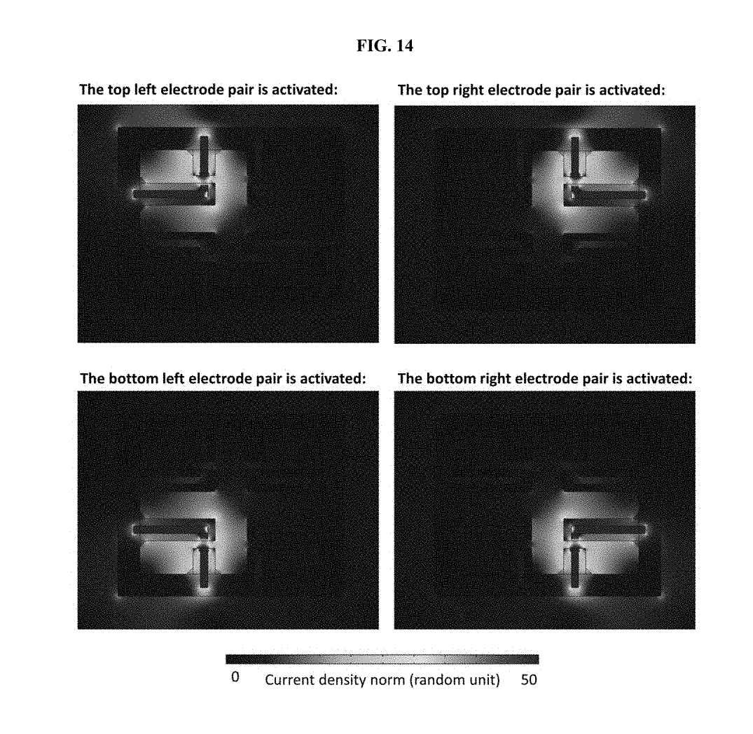

We have studied the response of our hydrogel ionic circuits to different mechanical inputs, including bending, stretching and pressing, using a simple circuit design consisting of one channel filled with saturated sodium sulfate solution (FIGS. 2A-2C). The hydrogel ionic circuits were generally more sensitive (higher channel resistance change) to stretching and pressing, due to larger geometric changes of the salt channels. The hydrogel ionic circuits were also stable when subjected to cyclic mechanical stimulation (FIG. 12).

Taking advantage of the re-programmability of the hydrogel ionic circuits, we designed a hydrogel LED display device that could be activated by mechanical press or touch. This device was prepared with PEG precursor solution containing 20% w/w PEGDMA 8,000 and 1% w/w Irgacure 2959 to achieve low stiffness. FIG. 2D shows a device with single LED. The power channel and the ground channel were connected with a shunt channel, which created a bypass for current when it was not pressed, so no current flowed through the LED in the resting state (FIG. 2E). When the shunt channel was pressed, the channel resistance dramatically increased, forcing some current to flow through LED/PEG hydrogel and activating the LED, which indicated a touch event (FIG. 2F). Expanding this single LED touch sensor into a 2-by-3 array (6 LEDs) allowed us to visually detect the touch position (FIG. 2G, FIG. 13). The flexible nature of the hydrogel ionic circuits allowed this LED touch panel to be mounted on curvilinear surfaces like human skin for wearable applications.

To test the utility of certain provided hydrogel ionic circuits in a biological context, we designed a hydrogel ionic electrode array for localized stimulation of in vitro cultured cells (FIGS. 3A, 3B). The electrode array consisted of 4 pairs of electrodes that were able to deliver electrical current to four locations by activating different electrode pairs (FIG. 14).

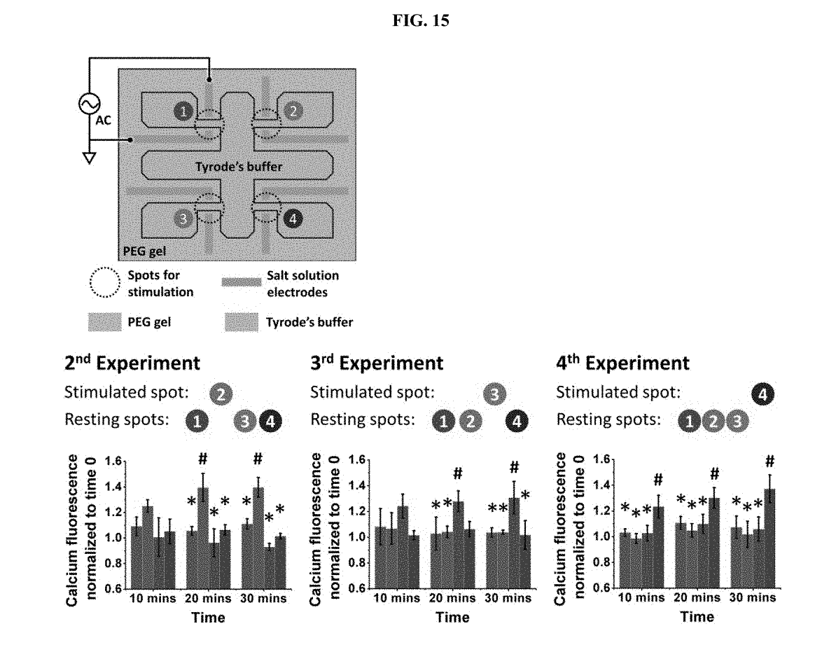

PEG hydrogel with 20% w/w PEGDMA 8,000, 20% w/w PEGDA 700, and 1% w/w irgacure 2959 was used for the fabrication of this device to achieve acceptable phase separation with Tyrode's solution. COMSOL simulations (FIG. 14) showed that the current was concentrated within the salt solution channels and crossed the PEG and Tyrode's buffer gaps at the narrowest part, so cells sitting in these gaps would be stimulated. The crosstalk between different stimulation spots due to current leakage through the PEG hydrogel was small, lower than 10% indicated by the simulation results. In cell experiments, human-derived neuroblastoma cells (SH-SY5Y) were seeded on the bottom of a OneWell cell culture plate in Tyrode's buffer, and the hydrogel electrode array was placed on top of the cells. To demonstrate regional activation with the device, one stimulation spot was activated using a pulsed signal at 1 Hz with a 2 millisecond pulse width and a field strength of 3.6 V/cm, while all other spots remained at rest. The experiment was repeated four times, activating a different spot each time. Cells were stimulated for 30 minutes and calcium staining (Fluo-4) was used to quantify the intracellular calcium concentration changes in response to the electrical stimulation. The fluorescence of all cells in a microscopic image was summed to obtain total fluorescence increase. The results showed that cells at the stimulated spots exhibited higher intracellular calcium increase compared to cells located at the resting spots (FIG. 3C, FIG. 15). This experiment demonstrated the cytocompatibility and usefulness of these hydrogel ionic circuits to deliver localized electrical stimulation in a biological environment.

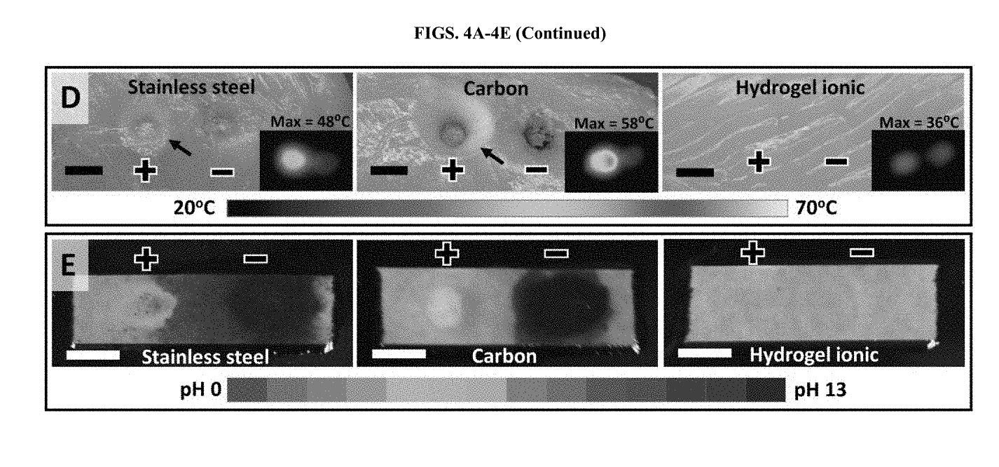

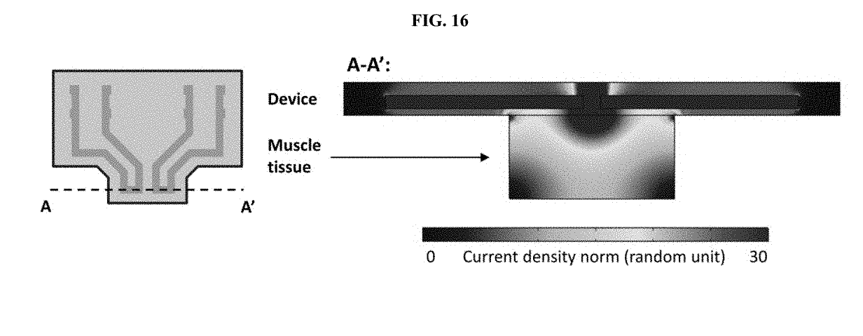

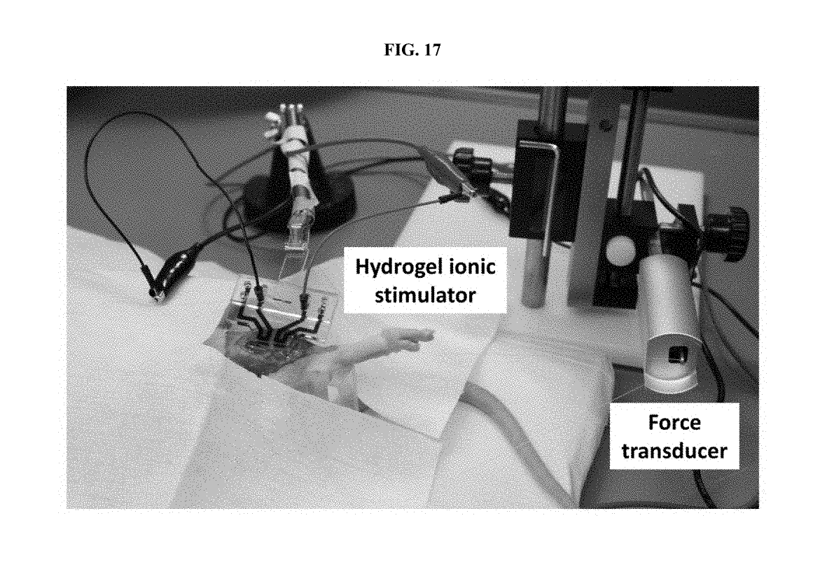

To confirm the ability of these hydrogel ionic circuits to form seamless bio-interfaces with soft tissues and effect in vivo electrical stimulation, skeletal muscle tissue stimulation experiments were conducted. A hydrogel ionic stimulator was designed with one pair of electrodes (FIG. 4A, FIG. 16). The device was prepared with 20% w/w PEGDMA 8,000 and 1% w/w Irgacure and was only partially covered with acrylic board, exposing the tip. The knees of Sprague Dawley rats were fixed using a custom-built platform and their tibialis anterior (TA) muscle was surgically exposed for electrical stimulation (FIG. 17). The hydrogel ionic stimulator was placed on the exposed muscle, which conformed to the tissue surface as a result of the mechanical compliance of the material, which also maximized the area of tissue available for stimulation (FIG. 4B). The TA was electrically stimulated at 1 Hz or 50 Hz with 2 millisecond or 40 microsecond pulse width and 0.9 V to 4.0 V voltage (the field strength was 4.5 to 20 V/cm) to measure twitch and tetanic forces, respectively. The force generation at 1 Hz increased slightly from 300 mN at a stimulation voltage of 0.9 V to plateau at 380 mN with stimulation voltages of either 1.6 or 2.5 V (FIG. 4C). There was a step-wise increase in the tetanic force measured at 50 Hz for the hydrogel ionic electrodes from 166 mN at a stimulation voltage of 0.9 V to 1.38 N at a stimulation voltage of 2.5 V (FIG. 4C). The force generated using a stimulation voltage of 2.5 V was within the ten-fold range for the tetanic strength of rat TA reported in the literature (0.80-10.7 N), and any differences with specific studies are likely a result of differences in the setup of the stimulation experiment or the fixation of the knee during testing (25, 26). As a control, we also stimulated the TA with a conventional bipolar gold stimulator used in a commercial device. A step-wise increase in the force measurements was observed for both twitch (1 Hz) and tetanic (50 Hz) force values. Interestingly, the hydrogel ionic stimulator stimulated statistically stronger twitch forces at a stimulation voltage of 1.6 V and statistically stronger tetanic forces at 1.6 and 2.5 V. To determine what stimulation voltage would generate tetanic forces comparable to the hydrogel electrode, the TA was stimulated with the conventional bipolar gold electrodes at 4 V. The tetanic force produced at 4 V with the bipolar gold electrodes (1.33 N) was similar to the force produced with the hydrogel ionic stimulator at V (1.38 N). Surprisingly, these data indicate that the hydrogel ionic stimulator was more effective at transmitting sub-tetanic signals to skeletal muscle, which may be advantageous in clinical scenarios where twitch forces are desired. Also surprising, it is of note that the hydrogel ionic stimulator was also more efficient than conventional metal electrodes in generating tetanic forces, being able to generate full tetanic contractions with a lower stimulation voltage. Together, these data suggest that in vivo, hydrogel ionic electrodes can transmit electrical signals to skeletal muscle tissue more efficiently than standard electrodes.

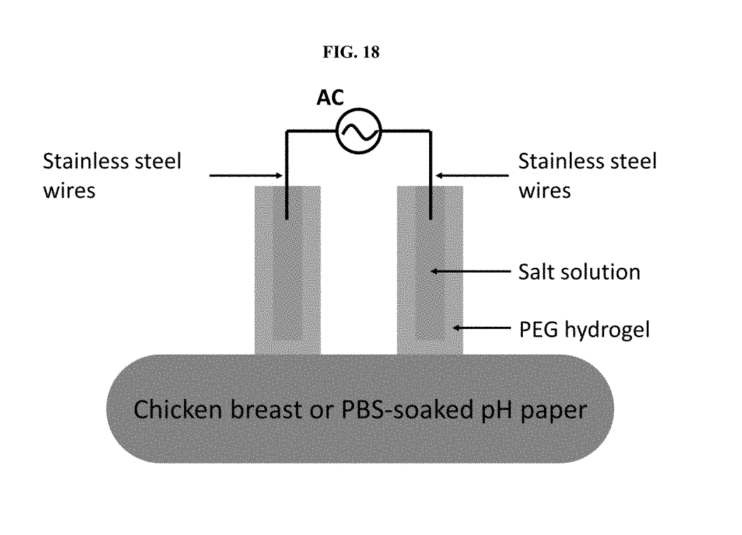

The injection of electrical current in tissues using conventional electron-conducting electrodes inevitably involves electrochemical reactions at electrode/tissue interface. These reactions induce chemical changes at the tissue site, such as the production of hydronium or hydroxide ions that changes the local pH, which together with charge injection-induced local heating can cause tissue damage (27-29). Here we demonstrate that, in some embodiments, provided hydrogel ionic stimulators reduced these adverse effects due to the high water content of the devices, which dissipates heat, as well as the potential use of pH buffering salts as the salt phase. We injected a constant current of 65 mA at a density of 0.72 A/cm² into an excised chicken breast for 30 seconds, which is significantly higher than the pain threshold for both dry (0.13 mA/cm²) and hydrogel electrodes (1.38 mA/cm²), but is relevant to the current density applied during electroporation and external defibrillation (16, 30, 31). Hydrogel ionic electrodes, carbon and stainless steel electrodes were tested (FIG. 18). Clear lesions were observed after current injection on the chicken breasts stimulated with carbon and stainless steel electrodes (FIG. 4D, left and middle panels, black arrows), but not on the chicken breast stimulated with the hydrogel ionic electrodes (FIG. 4D, right panel). The surface temperature profile was assessed immediately after current injection using an infrared camera (FLIR), showing milder increase of surface temperature of the chicken breast stimulated with hydrogel ionic electrodes (13° C. increase from room temperature) and higher local temperature beneath the carbon (35° C. increase from room temperature) and stainless steel (25° C. increase from room temperature) electrodes, consistent with the observation of lesions.

To reduce the pH changes from the current injection induced by electrochemical reactions at the metal electrode/salt solution interface, saturated sodium dibasic phosphate solution was used as the salt phase in the hydrogel ionic electrodes. Current was injected into phosphate buffered saline (PBS)-soaked pH papers for easy assessment of pH changes (FIG. 18). While there was no change in pH observed on the surface of the pH paper after 30 seconds of stimulation with 65 mA constant current at a density of 0.72 A/cm² using hydrogel ionic electrodes, there were dramatic differences in the pH when stainless steel and carbon electrodes were used (FIG. 4E). Without wishing to be held to a particular theory, these data suggest that using salt buffers in the hydrogel ionic electrodes can ameliorate pH changes at the point of electrical stimulation, making them safer to use in biological and clinical settings. Taking advantage of the unique salt/PEG aqueous two-phase system, these novel provided hydrogel ionic circuits offer new opportunities for biologically-matched electronic systems. These soft and completely aqueous-based devices can form seamless interfaces with biological tissues and optimize signal transduction while reducing local tissue damage. PEG hydrogels can be engineered to possess in vivo degradability through the use of enzyme-labile crosslinkers, which is required for many long-term in vivo utilities (32). Moreover, the high water content and transparency could enable optical and sonic camouflage of the devices (33). These devices can be potentially utilized for a broad range of applications from skin-mounted electrotactic stimulators to implantable pacemakers/defibrillators.

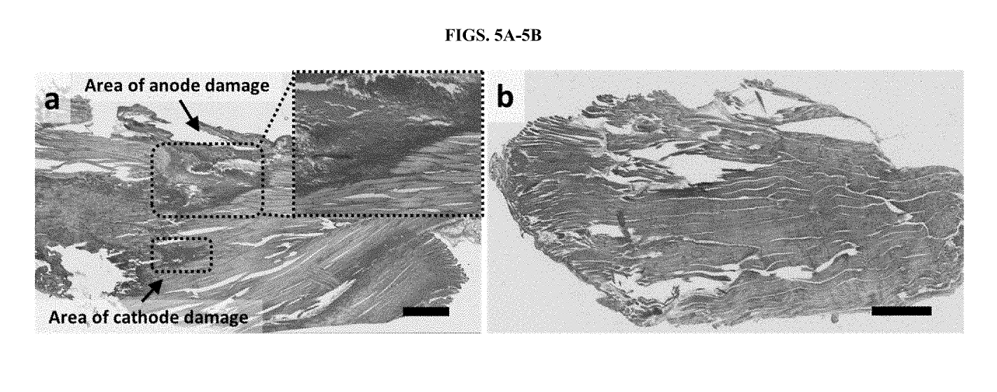

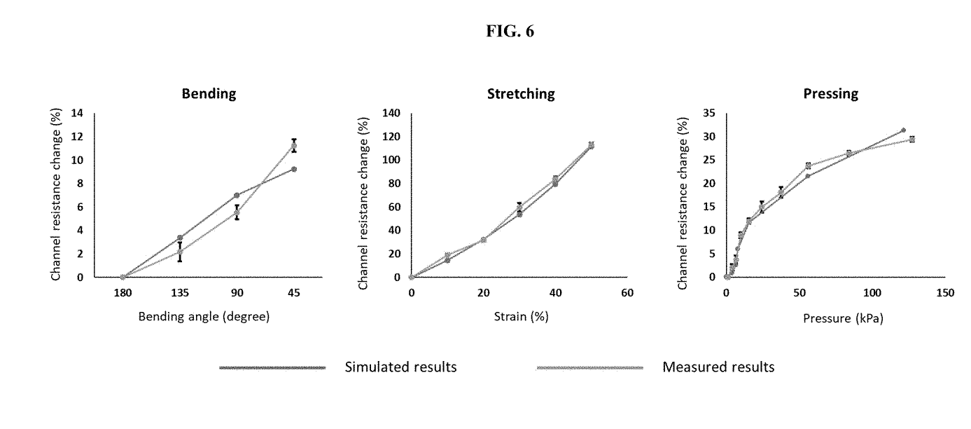

Data presented in FIGS. 5A-5B support a conclusion that hydrogel ionic circuits are able to reduce adverse effects associated with stimulation (see also FIG. 4D) by providing a closer, microscopic view at rat tibialis anterior muscle tissues that were stimulated using hydrogel ionic electrodes. The results were compared to conventional gold electrodes to show differences. Muscle tissues were stained with hematoxylin and eosin to show microscopic structures and striations.

The simulated results were consistent with measured values. FIG. 6 shows finite-element simulations of the experiments reported in FIGS. 2A-2C. COMSOL was used for the simulations. The simulation results show consistency with experimental results.

Materials and Methods

Materials and Device Fabrication

Polyethylene glycol dimethacrylate (PEGDMA, molecular weight: 8,000) was purchased from Polysciences (Warrington, Pa., USA). Polyethylene glycol diacrylate (PEGDA, molecular weight: 700), Irgacure 2959, benzophenone, sodium sulfate and sodium dibasic phosphate were purchased from Sigma Aldrich (St. Louis, Mo., USA). Sylgard 184 silicone elastomer kit (PDMS) was purchased from Fisher Scientific (Pittsburgh, Pa., USA). LEDs were purchased from Mouser electronics (Mansfield, Tex., USA). Liquid powder dye (Rit liquid dye) was purchased from local Walmart (Saugus, Mass., USA). Acrylic sheets and very-high-bond (VHB) foam tape were purchased from Mcmaster-Carr (Robbinsville, N.J., USA). Dulbecco's modified eagle medium with nutrient mixture F12 (DMEM/12), fetal bovine serum (FBS), penicillin-streptomycin and Fluo-4 AM calcium stain were purchased from Thermo Fisher Scientific (Grand Island, N.Y., USA). SH-SY5Y cells were purchased from ATCC (Manassas, Va., USA).

The 3-LED display devices, 3-by-5 LED display devices, one of the single-channel devices for cyclic press test, hydrogel ionic electrode array for SH-SY5Y stimulation, hydrogel ionic electrodes for chicken breast stimulation and pH characterization were fabricated using precursor containing 20% w/w PEGDMA 8 k, 20% w/w PEGDA 700, and 1% w/w irgacure 2959. The single-LED device for stretchability demonstration, single-channel devices for testing mechanical responses, one of the single-channel devices for cyclic press test, the LED touch sensors (single and 2-by-3) and the hydrogel ionic stimulators for in vivo muscle stimulation were fabricated using precursor containing 20% w/w PEGDMA 8 k and 1% w/w irgacure 2959.

To fabricate hydrogel ionic circuit devices, PDMS molds with desired conductive patterns (channels for salt solution perfusion) were first created. The patterns were subsequently transferred to PEG hydrogels using photo-crosslinking (34). The PEG hydrogel with channel patterns was bonded to a flat PEG hydrogel using photo-crosslinking to close the channels. It is important to avoid over-exposure when making the molded and the flat PEG hydrogels in order to ensure good bonding strength (35). Acrylic boards of 1.6 mm thick was used to cover the top and bottom of hydrogel ionic circuits to prevent water evaporation. The acrylic boards were coated with a layer of 0.5 mm thick VHB tape, which was treated with 10% w/w benzophenone in ethanol for 2 minutes to ensure good bonding with PEG hydrogels (36). Access holes were laser cut on the top acrylic board for easy fluid injection and electrical connections. The channels were perfused with salt solution to establish paths with high conductivity.

Device Characterization

To evaluate the long-term stability of salt/PEG phase separation, photocrosslinked PEG hydrogel discs were soaked in various ionic media and their resistivity was tested before soaking and at days 3, 7, 10, and 14. Four PEG hydrogel formulas were tested: 1) 5% w/w PEGDMA 8 k and 1% w/w irgacure 2959; 2) 20% w/w PEGDMA 8 k and 1% w/w irgacure 2959; 3) 20% w/w PEGDMA 8 k, 20% w/w PEGDA 700 and 1% w/w irgacure 2959; and 4) 20% w/w PEGDMA 8 k, 40% w/w PEGDA 700 and 1% w/w irgacure 2959. PEG hydrogels based on formula 1were tested with Tyrode's buffer, 5% w/w Na₂HPO₄ solution, DMEM and 5% w/w Na₂SO₄ solution to demonstrate that two-phase cannot be formed if the concentrations of PEG in hydrogels and/or the concentration of ionic media do not exceed threshold. PEG hydrogels based on formula 2-4 were tested with Tyrode's buffer, saturated Na₂HPO₄ solution, DMEM and saturated Na₂SO₄ solution to demonstrate successful phase separation. The resistivity of the hydrogel discs and the media were obtained using an Agilent 4284A LCR meter.

To assess the transparency of the PEG materials, 1 mm thick PEG hydrogel discs were fabricated at the bottom of a 96-well plate. The optical absorbance was measured from 400 to 700 nm at 50 nm intervals using a plate reader (SpectraMax M2, Molecular Devices). The transmittance was calculated from the absorbance (percent transmittance=10^{(2-absorbance)}).

To characterize the responses of hydrogel ionic circuits to mechanical stimulations, devices with single salt channel were pressed using a rheometer (TA instruments), stretched or bent, and the channel resistance changes were recorded using the LCR meter. The compressive strength of PEG hydrogels was measured using a universal mechanical testing system (Instron 3366).

In Vitro Cellular Electrical Stimulation using Hydrogel Ionic Electrode Array

SH-SY5Y cells were cultured in DMEM/F12 supplemented with 10% FBS and 1% antibiotics at 37° C., 5% CO₂. One day prior to experiment, SH-SY5Y cells were trypsinized and transferred to an OneWell tissue culture plate (Greiner) at 80,000 cells/cm². The cells were cultured for at least 24 hours to allow sufficient attachment. On the day of experiment, the cells were stained with Fluo-4 AM calcium dye (2.5 μg/ml in serum-free cell medium) for 45 minutes.

After staining, the cells were rinsed with Tyrode's buffer, which was also used as the buffer for the following stimulation experiments. The cells were stimulated with a positive-only pulsed signal with 1 Hz frequency, 2 millisecond pulse width and 3.6 V/cm field strength. The pulsed signal was generated from a data acquisition system (USB-6221, National Instruments) and amplified using a custom-built power amplifying circuit. The cells were stimulated for 30 minutes and the changes of intracellular calcium concentrations at both stimulated spots and unstimulated spots were monitored using fluorescent microscopy (Keyence).

In Vivo Muscle Electrical Stimulation using Hydrogel Ionic Stimulator

All animal protocols were approved by the Institutional Animal Care and Use Committee (IACUC) at Tufts University. Male Sprague Dawley rats (300 grams) were purchased from Charles River Laboratories. The rats were anesthetized with isoflurane (3 to 5%) and their hind legs were shaved and prepped for surgery. Animals were placed in the prone position on a customized operating table enabling the fixation of the knee joint. The tibialis anterior (TA) muscles were exposed via a skin incision with a scalpel blade. Bipolar hydrogel ionic stimulators and standard gold electrodes (control) with a 2 mm electrode separation were used to electrically stimulate the muscle tissues. The stimulation signals (1 Hz positive-only pulsed signal with 2 millisecond pulse width or 50 Hz positive-only pulsed signal with 40 microsecond pulse width, with a voltage ranging from 0.9 V to 4.0 V) were generated from a data acquisition system (USB-6221, National Instruments) and amplified using a custom-built power amplifying circuit.

Functional assessment of the hydrogel ionic stimulators and gold electrodes to stimulate muscle tissues was conducted by measuring twitch (1 Hz) and tetanic (50 Hz) forces. Prior to stimulation the foot was anchored at the cleft between digits 1 and 2 to a force transducer using nylon ligature. Either the hydrogel ionic stimulator or gold electrodes were placed in contact with the exposed muscle to stimulate contraction. The force of each contraction was measured and recorded using LabChart 7 (ADinstruments).

Statistical Analysis

IBM SPSS Statistics 22 Software (New York, USA) was used to perform One Way ANOVA for statistical analysis (p<0.05) in order to evaluate the significance of localized electrical stimulation for in vitro cell cultures. Post-hoc comparison of means was performed by Tukey HSD test, and post-hoc procedures and statistical significance were considered at p<0.05. All statistics were performed using 3 replicates. To evaluate the ability of electrodes to stimulate muscle contraction in vivo, a Student's t-test was performed between the two electrode types (hydrogel ionic and metal). Differences between the conditions were considered significant at p<0.05 (n≥1).

### REFERENCES

1. D. McCoul, W. Hu, M. Gao, V. Mehta, Q. Pei, Recent advances in stretchable and transparent electronic materials. Adv. Electron. Mater. 2, 1500407 (2016).
2. H. Wu, W. Gao, Z. Yin, Materials, devices and systems of soft bioelectronics for precision therapy. Adv Healthc Mater 6, 1700017 (2017).
3. B. Xu, A. Akhtar, Y. Liu, H. Chen, W. H. Yeo, S. I. Park, B. Boyce, H. Kim, J. Yu, H. Y. Lai, S. Jung, Y. Zhou, J. Kim, S. Cho, Y. Huang, T. Bretl, J. A. Rogers, An epidermal stimulation and sensing platform for sensorimotor prosthetic control, management of lower back exertion, and electrical muscle activation. Adv Mater 28, 4462-4471 (2016).
4. H. Fang, K. J. Yu, C. Gloschat, Z. Yang, C. H. Chiang, J. Zhao, S. M. Won, S. Xu, M. Trumpis, Y. Zhong, E. Song, S. W. Han, Y. Xue, D. Xu, G. Cauwenberghs, M. Kay, Y. Huang, J. Viventi, I. R. Efimov, J. A. Rogers, Capacitively coupled arrays of multiplexed flexible silicon transistors for long-term cardiac electrophysiology. Nat Biomed Eng 1, 0038 (2017).
5. M. M. Hamedi, A. Ainla, F. Guder, D. C. Christodouleas, M. T. Fernandez-Abedul, G. M. Whitesides, Integrating electronics and microfluidics on paper. Adv Mater 28, 5054-5063 (2016).
6. D. H. Kim, N. Lu, R. Ma, Y. S. Kim, R. H. Kim, S. Wang, J. Wu, S. M. Won, H. Tao, A. Islam, K. J. Yu, T. I. Kim, R. Chowdhury, M. Ying, L. Xu, M. Li, H. J. Chung, H. Keum, M. McCormick, P. Liu, Y. W. Zhang, F. G. Omenetto, Y. Huang, T. Coleman, J. A. Rogers, Epidermal electronics. Science 333, 838-843 (2011).
7. D. H. Kim, J. Viventi, J. J. Amsden, J. Xiao, L. Vigeland, Y. S. Kim, J. A. Blanco, B. Panilaitis, E. S. Frechette, D. Contreras, D. L. Kaplan, F. G. Omenetto, Y. Huang, K. C. Hwang, M. R. Zakin, B. Litt, J. A. Rogers, Dissolvable films of silk fibroin for ultrathin conformal bio-integrated electronics. Nat Mater 9, 511-517 (2010).
8. J. Xu, S. Wang, G. N. Wang, C. Zhu, S. Luo, L. Jin, X. Gu, S. Chen, V. R. Feig, J. W. To, S. Rondeau-Gagne, J. Park, B. C. Schroeder, C. Lu, J. Y. Oh, Y. Wang, Y. H. Kim, H. Yan, R. Sinclair, D. Zhou, G. Xue, B. Murmann, C. Linder, W. Cai, J. B. Tok, J. W. Chung, Z. Bao, Highly stretchable polymer semiconductor films through the nanoconfinement effect. Science 355, 59-64 (2017).
9. X. Pu, H. Guo, J. Chen, X. Wang, Y. Xi, C. Hu, Z. L. Wang, Eye motion triggered self-powered mechnosensational communication system using triboelectric nanogenerator. Sci Adv 3, e1700694(2017).
10. X. Pu, M. Liu, X. Chen, J. Sun, C. Du, Y. Zhang, J. Zhai, W. Hu, Z. L. Wang, Ultrastretchable, transparent triboelectric nanogenerator as electronic skin for biomechanical energy harvesting and tactile sensing. Sci Adv 3, e1700015 (2017).
11. Y. Wang, C. Zhu, R. Pfattner, H. Yan, L. Jin, S. Chen, F. Molina-Lopez, F. Lissel, J. Liu, N. I. Rabiah, Z. Chen, J. W. Chung, C. Linder, M. F. Toney, B. Murmann, Z. Bao, A highly stretchable, transparent, and conductive polymer. Sci Adv 3, e1602076 (2017).
12. C. Keplinger, J. Y. Sun, C. C. Foo, P. Rothemund, G. M. Whitesides, Z. Suo, Stretchable, transparent, ionic conductors. Science 341, 984-987 (2013).
13. C. H. Yang, B. Chen, J. J. Lu, J. H. Yang, J. Zhou, Y. M. Chen, Z. Suo, Ionic cable. Extreme Mechanics Letters 3, 59-65 (2015).
14. I. Noshadi, B. W. Walker, R. Portillo-Lara, E. Shirzaei Sani, N. Gomes, M. R. Aziziyan, N. Annabi, Engineering biodegradable and biocompatible bio-ionic liquid conjugated hydrogels with tunable conductivity and mechanical properties. Sci Rep 7, 4345 (2017).
15. B. Song, Y. Gu, J. Pu, B. Reid, Z. Zhao, M. Zhao, Application of direct current electric fields to cells and tissues in vitro and modulation of wound electric field in vivo. Nat Protoc 2, 1479-1489 (2007).
16. H. Zhou, Y. Lu, W. Chen, Z. Wu, H. Zou, L. Krundel, G. Li, Stimulating the Comfort of Textile Electrodes in Wearable Neuromuscular Electrical Stimulation. Sensors (Basel) 15, 17241-17257 (2015).
17. J. Y. Sun, C. Keplinger, G. M. Whitesides, Z. Suo, Ionic skin. Adv Mater 26, 7608-7614 (2014).
18. H. Yuk, T. Zhang, G. A. Parada, X. Liu, X. Zhao, Skin-inspired hydrogel-elastomer hybrids with robust interfaces and functional microstructures. Nat Commun 7, 12028 (2016).
19. C. C. Kim, H. H. Lee, K. H. Oh, J. Y. Sun, Highly stretchable, transparent ionic touch panel. Science 353, 682-687(2016).
20. S. Hardt, T. Hahn, Microfluidics with aqueous two-phase systems. Lab Chip 12, 434-442 (2012).
21. A. Glyk, T. Scheper, S. Beutel, PEG-salt aqueous two-phase systems: an attractive and versatile liquid-liquid extraction technology for the downstream processing of proteins and enzymes. ApplMicrobiol Biotechnol 99, 6599-6616 (2015).
22. H. Tavana, A. Jovic, B. Mosadegh, Q. Y. Lee, X. Liu, K. E. Luker, G. D. Luker, S. J. Weiss, S. Takayama, Nanolitre liquid patterning in aqueous environments for spatially defined reagent delivery to mammalian cells. Nat Mater 8, 736-741 (2009).
23. M. T. Zafarani-Moattar, R. Sadeghi, Liquid—liquid equilibria of aqueous two-phase systems containing polyethylene glycol and sodium dihydrogen phosphate or disodium hydrogen phosphate Experiment and correlation. Fluid Phase Equilibria 181, 95-112 (2001).
24. L. A. Ferreira, J. A. Teixeira, Salt effect on the aqueous two-phase system peg 8000-sodium sulfate. J. Chem. Eng. Data 56, 133-137 (2011).
25. K. Garg, B. T. Corona, T. J. Walters, Losartan administration reduces fibrosis but hinders functional recovery after volumetric muscle loss injury. J Appl Physiol 117, 1120-1131 (2014).
26. B. Kasukonis, J. Kim, L. Brown, J. Jones, S. Ahmadi, T. Washington, J. Wolchok, Codelivery of infusion decellularized skeletal muscle with minced muscle autografts improved recovery from volumetric muscle loss injury in a rat model. Tissue Eng Part A 22, 1151-1163 (2016).
27. D. R. Merrill, M. Bikson, J. G. Jefferys, Electrical stimulation of excitable tissue: design of efficacious and safe protocols. J Neurosci Methods 141, 171-198 (2005).
28. D. Liebetanz, R. Koch, S. Mayenfels, F. Konig, W. Paulus, M. A. Nitsche, Safety limits of cathodal transcranial direct current stimulation in rats. Clin Neurophysiol 120, 1161-1167 (2009).
29. U. Pliquett, Joule heating during solid tissue electroporation. Med Biol Eng Comput 41, 215-219 (2003).
30. I. Serša, M. Kranjc, D. Miklavčič, Current density imaging sequence for monitoring current distribution during delivery of electric pulses in irreversible electroporation. BioMedical Engineering OnLine 14 (suppl 3), (2015).
31. V. T. Krasteva, S. P. Papazov, Estimation of current density distribution under electrodes for external defibrillation. Biomed Eng Online 1, 7 (2002).
32. E. B. Peters, N. Christoforou, K. W. Leong, G. A. Truskey, J. L. West, Poly(ethylene glycol) hydrogel scaffolds containing cell-adhesive and protease-sensitive peptides support microvessel formation by endothelial progenitor cells. Cell Mol Bioeng 9, 38-54 (2016).
33. H. Yuk, S. Lin, C. Ma, M. Takaffoli, N. X. Fang, X. Zhao, Hydraulic hydrogel actuators and robots optically and sonically camouflaged in water. Nat Commun 8, 14230 (2017).
34. H. Cong, A. Revzin, T. Pan, Non-adhesive PEG hydrogel nanostructures for self-assembly of highly ordered colloids. Nanotechnology 20, 075307 (2009).
35. P. Kim, H. E. Jeong, A. Khademhosseini, K. Y. Suh, Fabrication of non-biofouling polyethylene glycol micro- and nanochannels by ultraviolet-assisted irreversible sealing. Lab Chip 6, 1432-1437 (2006).
36. H. Yuk, T. Zhang, G. A. Parada, X. Liu, X. Zhao, Skin-inspired hydrogel-elastomer hybrids with robust interfaces and functional microstructures. Nat Commun 7, 12028 (2016).
37. F. L. Gielen, W. Wallinga-de Jonge, K. L. Boon, Electrical conductivity of skeletal muscle tissue: experimental results from different muscles in vivo. Med Biol Eng Comput 22, 569-577 (1984).

We claim:

1. A hydrogel ionic circuit comprising
a molded, crosslinked polyethylene glycol (PEG) hydrogel polymer comprising at least two electrode channels separated by a gap, wherein the channels comprise a salt solution; and
at least two ports to connect the salt solution electrode channels to a power source.

2. The circuit of claim 1, wherein the PEG hydrogel polymer comprises at least 15% by weight of a high molecular weight PEG.

3. The circuit of claim 2, wherein the PEG hydrogel polymer comprises at least 20% by weight of a high molecular weight PEG.

4. The circuit of claim 2, wherein the PEG is polyethylene glycol dimethacrylate molecular weight 8,000 (PEGMA 8 k).

5. The circuit of claim 2, wherein the PEG hydrogel polymer additionally comprises at least 15% by weight of a low molecular weight PEG.

6. The circuit of claim 5, wherein the PEG hydrogel comprises at least 20% by weight of polyethylene glycol diacrylate molecular weight 700 (PEGDA 700).

7. The circuit of claim 1, wherein the salt solution is a sodium sulfate (Na₂SO₄) solution or a sodium phosphate (Na₂HPO₄) solution.

8. The circuit of claim 1, wherein the salt solution is a saturated salt solution.

9. The circuit of claim 1, wherein the circuit additionally comprises an electronically responsive component, wherein when a voltage difference is applied between the salt solution electrode channels, an induced current will activate the electronically responsive component.

10. The circuit of claim 9, wherein the electronically responsive component is a light emitting diode (LED) or organic light emitting diode (OLED).

11. The circuit of claim 1, wherein the circuit additionally comprises one or more cells in the gap between the at least two electrode channels.

12. The circuit of claim 1, wherein at least two faces of the circuit are covered to prevent water evaporation, wherein the ports extend through the cover.

13. The circuit of claim 1, wherein the circuit additionally comprises an aqueous-based connective interface at the gap between the salt solution electrode channels.

14. A device comprising one or more hydrogel ionic circuits of claim 1 and a power source.

15. The device of claim 14, wherein the device comprises a light-emitting diode (LED) or organic light emitting diode (OLED).

16. The device of claim 15, wherein the device comprises an aqueous-based connective interface at the gap between the salt solution electrode channels in the circuit.

17. A method of stimulating tissue comprising the steps of:
contacting the aqueous-based connective interface of the circuit of claim 13 to a tissue; and
applying a voltage difference across the salt solution electrode channels of the circuit, whereby the induced current stimulates the tissue.

18. The method of claim 17, wherein the tissue is a tissue in a subject and the circuit is implanted into the subject.

19. A method for fabricating a hydrogel ionic circuit comprising the steps of:
providing a solution comprising at least 15% by weight of a high molecular weight PEG and between about 0.005% and about 5.0% by weight of a photoinitiator on a mold with a raised or grooved channel pattern;
photocrosslinking the high molecular weight PEG by exposure to ultra-violet (UV) light to form a PEG hydrogel with a channel pattern;
bonding the PEG hydrogel with a channel pattern to a flat PEG hydrogel by exposure to UV light; and
introducing a salt solution into channels of the PEG hydrogel via a port.

20. The method of claim 19, wherein one or more light-emitting diodes (LEDs) are added between the PEG hydrogel with a channel pattern and the flat PEG hydrogel prior to bonding.
